(19) United States
(12) Patent Application Publication
(10) Pub. No.: US 2026/0356975 A1
(43) Pub. Date: Nov. 12, 2026

(54) METHODS AND PHARMACEUTICAL COMPOSITIONS FOR THE TREATMENT OF OSTEOARTHRITIS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); UNIVERSITE COTE D'AZUR, Nice (FR); CENTRE HOSPITALIER UNIVERSITAIRE DE NICE, Nice (FR); CENTRE HOSPITALIER UNIVERSITAIRE DE MONTPELLIER, Montpellier (FR); UNIVERSITÉ DE MONTPELLIER, Montpellier (FR)

(72) Inventors: Julien CHERFILS-VICINI, Nice Cedex 2 (FR); Eric GILSON, Nice Cedex 2 (FR); Christian JORGENSEN, Montpellier Cedex 5 (FR); Jean-Marc BRONDELLO, Montpellier Cedex 5 (FR); Yves-Marie PERS, Montpellier Cedex 5 (FR); Christina FISSOUN, Montpellier Cedex 5 (FR)

(21) Appl. No.: 19/113,552

(22) PCT Filed: Oct. 20, 2023
(86) PCT No.: PCT/EP2023/079262

§ 371 Date: Mar. 20, 2025

(30) Foreign Application Priority Data
Oct. 21, 2022 (EP) 22306604.4

(51) Int. Cl. C07K 16/30; A61K 39/00; A61K 45/06; A61P 19/02

(52) U.S. Cl. CPC C07K 16/3084; A61K 45/06; A61P 19/02; A61K 2039/505; C07K 2317/76

(57) ABSTRACT

Osteoarthritis (OA) is a type of degenerative joint disease that results from breakdown of joint cartilage and underlying bone. Here, the inventors studied the effects of intra-articular anti-GD3 monoclonal antibody injection. The present invention relates to a method for treating osteoarthritis in a subject in need thereof, comprising administering to said subject a therapeutically effective amount of a GD3 inhibitor.

### FIELD OF THE INVENTION

The present invention relates to methods and pharmaceutical compositions for the treatment of osteoarthritis.

### BACKGROUND OF THE INVENTION

Osteoarthritis (OA) is the most common degenerative joint disease among the rheumatic disorders affecting the Western world. This condition is one of the main causes of pain and incapacity in elderly people, becoming a major health problem.

Osteoarthritis is characterized by degeneration of the articular cartilage, remodelling of the subchondral bone and changes in the synovial membrane. It commonly affects the hands, feet, spine, and large extraspinal, weight-bearing joints, such as the hips and knees. The etiology of osteoarthritis is multifactorial involving both mechanical and biochemical factors. Clinical classification includes several phenotypes such as post-traumatic, metabolic, ageing or genetic.

Clinical manifestations of OA are joint pain, stiffness in the morning or after rest, pain at night, limited motion, joint deformity, associated with variable degrees of inflammation of the synovial membrane (synovitis). Joint pain in OA may originate not only from synovitis, but also from stretching of the joint capsule or ligaments, periosteal irritation, trabecular microfractures, intraosseous hypertension or muscle spasms.

Some treatments of osteoarthrosis are available, such as analgesics, nonsteroidal anti-inflammatory drugs, corticosteroids, hyaluronic acid injection, surgery (realigning bones or joint replacement), but these treatments are of high cost. Thus, there is a need for an improved method to treat osteoarthritis.

### SUMMARY OF THE INVENTION

The present invention relates to methods and pharmaceutical compositions for the treatment of osteoarthritis. In particular, the present invention is defined by the claims.

### DETAILED DESCRIPTION OF THE INVENTION

Osteoarthritis (OA) is a type of degenerative joint disease that results from breakdown of joint cartilage and underlying bone. Here, the inventors studied the effects of intra-articular anti-GD3 monoclonal antibody injection.

In a first embodiment, the present invention relates to a method for treating osteoarthritis in a subject in need thereof, comprising administering to said subject a therapeutically effective amount of a GD3 inhibitor.

As used herein, the terms “subject” or “patient” denote a mammal, such as a rodent, a feline, a canine, and a primate. Particularly, the subject according to the invention is a human. Particularly, the subject according to the invention is a child, a teenager, an adult or an elderly person. In some embodiments, the subject is more than 15 years old. In some embodiments, the subject is more than 20 years old. In some embodiments, the subject is more than 25 years old. In some embodiments, the subject is more than 30 years old. In some embodiments, the subject is more than 35 years old.

As used herein, the term “osteoarthritis” (OA) has its general meaning in the art and refers to a degenerative joint disease with moderate local inflammation occurring chiefly in older humans and animals, which is characterized by degeneration of the articular cartilage, remodelling of the subchondral bone and changes in the synovial membrane. Osteoarthritis can affect any joint of the organism, such as knee, hip, elbow, hands joints, shoulder, back (for example, spinal or cervical), foot joints, ankle, etc. Osteoarthritis can be a metabolic osteoarthritis, which is related to an accumulation of metabolic abnormalities or metabolic syndrome.

In one embodiment, osteoarthritis is metabolic osteoarthritis.

In one embodiment, osteoarthritis is post-traumatic osteoarthritis.

As used herein, the term “post-traumatic osteoarthritis” refers to inflammation in joints that forms after experienced a trauma. It develops quickly after an injury instead of over years of wear and tear like other forms of arthritis. Post-traumatic arthritis can last longer and becomes a chronic (long-term) condition.

As used herein, the term “ganglioside” refers to a molecule composed of a glycosphingolipid (ceramide and oligosaccharide) with one or more sialic acids (e.g. n-acetylneuraminic acid, NANA) linked on the sugar chain. NeuNAc, an acetylated derivative of the carbohydrate sialic acid, makes the head groups of gangliosides anionic at pH 7, which distinguishes them from globosides.

In some embodiment, the present invention relates to the inhibition of the enzyme ST8SIA1.

As used herein, the term “ST8SIA1” also known as “Alpha-N-acetylneuraminide alpha-2,8-sialyltransferase” has its general meaning in this art and Catalyzes the addition of sialic acid in alpha 2,8-linkage to the sialic acid moiety of the ganglioside GM3 to form ganglioside GD3 (Uniprot accession number Q92185).

As used herein, the term “inhibitor” as used herein includes not only drugs for inhibiting activity of target molecules, but also drugs for inhibiting the expression of target molecules.

As used herein, the term “GD3” has its general meaning in this art and is defined by the chemical structure: aNeu5Ac(2-8)aNeu5Ac(2-3)bDGalp(1-4)bDGlcp(1-1)Cer. In some aspects, the GD3 is human GD3. In some aspects, the GD3 is rat GD3. In some aspects, the GD3 is mouse GD3. In some aspects, the GD3 is primate GD3. GD3 includes variants, isoforms, homologs, orthologs and paralogs of human ganglioside GD3.

In some embodiment, the present invention relates to the inhibition of GD3.

As used herein, the term “GD3 inhibitor” has its general meaning in the art and refers to an inhibitor of GD3. The inhibitor of GD3 include but are not limited to GD3 antibody, CAR-T cell, CAR NK cell.

In some embodiment, the present invention relates to senotherapy strategy including any type of targeting (e.g. antibody, drug, CAR T, CAR NK . . . ) of GD3 that aimed at blocking with senomorphic agent or eliminating with senolytic agent senescent cells by targeting GD3.

In some embodiments, the GD3 inhibitor is an antibody.

As used herein, the term “antibody” is used in the broadest sense and specifically covers monoclonal antibodies, polyclonal antibodies, multi-specific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404,097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388. In a particular embodiment, the inhibitor is a monoclonal antibody.

Monoclonal antibodies can be prepared and isolated using any technique to produce antibody molecules by continuous cell lines in culture. Techniques for production and isolation include but are not limited to the hybridoma technique, the human B-cell hybridoma technique and the EBV-hybridoma technique. In a particular embodiment, the antibody is specific of the isoform B of GD3. In some embodiments, the antibody is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb. Sanofi-Genzyme has developed a blocking and depleting antibody SAR440241. R&D Systems has developed a blocking antibody MAB-160. This kind of antibodies affects mostly immune cells, notably the immune adaptive system and not melanocytes.

In a particular embodiment, the GD3 antibody includes but are not limited to the clone MB3.6 or the clone R24 or derivative from those clones (afucosylated, humanized . . . ). Examples of GD3 antibody includes but are not limited to Ganglioside GD3 Monoclonal Antibody of LifeSpan BioSciences, ST8 alpha-2,8-Sialyltransferase 8A/ST8SIA1/Ganglioside GD3 Antibody of Novus Biologicals, Ganglioside GD3 (2Q631) Antibody and Ganglioside GD3 (MB3.6) Antibody of Santa Cruz Biotechnology, Inc., Mab Mo x human Ganglioside GD3 antibody of United States Biological, Mitumomab, Ecromeximab and clone U36 of Creative Biolabs.

In a particular embodiment, the GD3 inhibitor is a CAR-T cell.

As used herein the term “CAR-T cell” refers to a T lymphocyte that has been genetically engineered to express a CAR. The definition of CAR T-cells encompasses all classes and subclasses of T-lymphocytes including CD4+, CD8+ T cells, gamma delta T cells as well as effector T cells, memory T cells, regulatory T cells, and the like. The T lymphocytes that are genetically modified may be “derived” or “obtained” from the subject who will receive the treatment using the genetically modified T cells or they may “derived” or “obtained” from a different subject. As used herein, the term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an antigen binding domain to an intracellular signaling domain. In some embodiments, the stimulatory molecule is the zeta chain associated with the T cell receptor complex. In some embodiments, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In particular aspects, CARs comprise fusions of single-chain variable fragments (scFv) derived from monoclonal antibodies, fused to CD3-zeta a transmembrane domain and endodomain. In some embodiments, CARs comprise domains for additional co-stimulatory signaling, such as CD3-zeta, FcR, CD27, CD28, CD137, DAPIO, and/or 0X40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

In a particular embodiment, the GD3 inhibitor is a CAR NK cell.

As used herein the term “CAR-NK” refers to natural killer (NK) cells that has been genetically engineered to express a CAR. NK cells are defined as CD56+ and CD3− cells and are subdivided into cytotoxic and immunoregulatory. They are of great clinical interest because they contribute to the graft-vs-leukemia/graft-vs-tumor effect but are not responsible for graft-vs-host disease. NK cells can be generated from various sources such as umbilical cord blood, bone marrow, human embryonic stem cells, and induced pluripotent stem cells. However, tumors can escape the cytotoxicity of NK cells when they are directed against NKG2D ligands MICA and MICB (major histocompatibility complex class I chain-related protein A/B). Henceforth, preclinical research has been reported for CAR-modified primary human NK cells redirected against CD19, CD20, CD244, and HER2, as well as CAR-expressing NK-92 cells targeted to a wider range of cancer antigens. Primary NK cells engineered to express CARs have potential benefits compared to CAR-T cells. NK cells have spontaneous cytotoxic activity and can generate target cell death independent of tumor antigen, while T lymphocytes only kill their targets by a CAR-specific mechanism. Therefore, in the setting of antigen downregulation by tumor cells attempting to escape immune detection, NK cells would still be effective against tumor cells. In addition, primary human NK cells produce cytokines, such as interferon gamma, interleukin 3, and granulocyte-macrophage colony-stimulating factor, that differ from the proinflammatory cytokines produced by T cells that are responsible for the onset of cytokine release syndrome. Individual NK cells can survive after contacting and killing multiple target cells, possibly reducing the number of cells that need to be adoptively transferred (ie, the ex vivo stimulation and expansion of autologous or allogeneic lymphocytes, followed by reinfusion of the expanded lymphocyte population into the patient, in contrast to T cells). Furthermore, whereas the long-term persistence of CAR-T cells may maintain on-target, off-tumor toxicity such as the B cell aplasia seen with anti-CD19 CAR-T cells, mature NK cells are short lived and are expected to disappear after facilitating their anticancer effects

In a particular embodiment, the GD3 inhibitor is a peptide, peptidomimetic, small organic molecule, antibody, aptamers, siRNA or antisense oligonucleotide. The term “peptidomimetic” refers to a small protein-like chain designed to mimic a peptide.

In a particular embodiment, the GD3 inhibitor is an aptamer.

The term “Aptamers” refer to a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., Science, 1990, 249(4968):505-10. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., Clin. Chem., 1999, 45(9):1628-50. Peptide aptamers consists of a conformationally constrained antibody variable region displayed by a platform protein, such as E. coli Thioredoxin A that are selected from combinatorial libraries by two hybrid methods (Colas et al., Nature, 1996, 380, 548-50).

In a particular embodiment, the GD3 inhibitor is a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In some embodiments, the GD3 inhibitor is a short hairpin RNA (shRNA), a small interfering RNA (siRNA) or an antisense oligonucleotide which inhibits the expression of GD3.

In a particular embodiment, the inhibitor of GD3 expression is siRNA

In a particular embodiment, the inhibitor of GD3 expression is shRNA.

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Ribozymes can also function as an inhibitor of GD3 expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mineralocorticoid receptor mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as Moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991.

Preferred viruses for certain applications are the adeno-viruses and adeno-associated viruses, which are double-stranded DNA viruses. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. These plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, intra-articular or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In some embodiments, the GD3 inhibitor is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error-prone nonhomologous end-joining (NHEJ) and the high-fidelity homology-directed repair (HDR).

In a particular embodiment, the endonuclease is CRISPR-cas.

As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671), zebrafish (Hwang et al., 2013, PLoS One, Vol. 8:e68708), C. elegans (Hai et al., 2014 Cell Res. doi: 10.1038/cr.2014.11.), bacteria (Fabre et al., 2014, PLoS Negl. Trop. Dis., Vol. 8:e2671), plants (Mali et al., 2013, Science, Vol. 339: 823-826), Xenopus tropicalis (Guo et al., 2014, Development, Vol. 141: 707-714), yeast (DiCarlo et al., 2013, Nucleic Acids Res., Vol. 41: 4336-4343), Drosophila (Gratz et al., 2014 Genetics, doi:10.1534/genetics.113.160713), monkeys (Niu et al., 2014, Cell, Vol. 156: 836-843), rabbits (Yang et al., 2014, J. Mol. Cell Biol., Vol. 6: 97-99), pigs (Hai et al., 2014, Cell Res. doi: 10.1038/cr.2014.11.), rats (Ma et al., 2014, Cell Res., Vol. 24: 122-125) and mice (Mashiko et al., 2014, Dev. Growth Differ. Vol. 56: 122-129). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In some embodiments, the GD3 inhibitor is an aptamer. In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the term “treatment” or “treat” refer to both prophylactic or preventive treatment as well as curative, improving the patient's condition or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., daily, weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “preventing” intends characterizing a prophylactic method or process that is aimed at delaying or preventing the onset of a disorder or condition to which such term applies.

As used herein the terms “administering” or “administration” refer to the act of injecting or otherwise physically delivering a substance as it exists outside the body (e.g. GD3 inhibitor) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen.

Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Accordingly, the subject is administered with a pharmaceutical composition comprising the GD3 inhibitor as active principle and at least one pharmaceutically acceptable excipient. As used herein the term “active principle” or “active ingredient” are used interchangeably. The active principle is used to alleviate, treat or prevent a medical condition or disease. By the term “pharmaceutically acceptable excipient” herein, it is understood a carrier medium which does not interfere with the effectiveness of the biological activity of the active ingredient(s) and which is not excessively toxic to the host at the concentration at which it is administered. Said excipients are selected, depending on the pharmaceutical form and the desired method of administration, from the usual excipients known by a person skilled in the art. In some embodiments, the pharmaceutical composition of the present invention does not comprise a second active principle.

As used herein, the term “combination” is intended to refer to all forms of administration that provide a first drug together with a further (second, third . . . ) drug. The drugs may be administered simultaneously, separately or sequentially and in any order. According to the invention, the drug is administered to the subject using any suitable method that enables the drug to reach the chondrocytes of the bone growth plate. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the growing bone.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In some embodiments, the GD3 inhibitor of the present invention is administered to the subject in combination with a standard treatment. For instance, standard treatment of osteoarthritis is analgesics, nonsteroidal anti-inflammatory drugs, corticosteroids, hyaluronic acid injection, surgery, physical activity, weight management, etc

In some embodiments, the treatment consists of administering to the subject an immunotherapeutic agent. The term “immunotherapeutic agent”, as used herein, refers to a compound, composition or treatment that indirectly or directly enhances, stimulates, or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies, and non-cytokine adjuvants. Alternatively, the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins, and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoetin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pegfilgrastim; Amgen), Leukine (GM-CSF; Berlex), Procrit (erythropoietin; Ortho Biotech), Epogen (erythropoietin; Amgen), Aranesp (erythropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

In some embodiments, the subject will be treated with a GD3 inhibitor in combination with an immune checkpoint inhibitor.

In some embodiments, the therapy consists of administering to the subject an immune checkpoint inhibitor in combination with GD3 inhibitor.

In a particular embodiment, i) an immune checkpoint inhibitor and ii) GD3 inhibitor as a combined preparation according to the invention for simultaneous, separate, or sequential use in the method for treating osteoarthritis in a subject.

As used herein, the term “immune checkpoint inhibitor” (ICI) refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e.g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-napthoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-napthoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophényl)-N′-hydroxy-4-{[2-(sulfamoylamino)-éthyl]amino}-1,2,5-oxadiazole-3 carboximidamide:

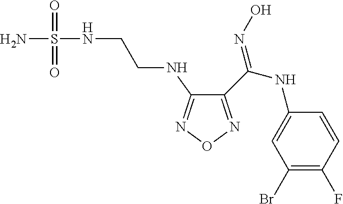 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

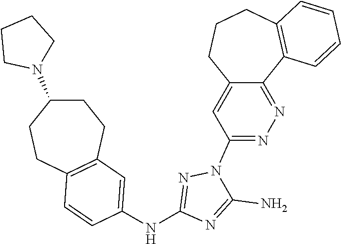 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

Typically, the GD3 inhibitor as described above are administered to the subject in the form of a pharmaceutical composition which comprises a pharmaceutically acceptable carrier.

The term “pharmaceutically” or “pharmaceutically acceptable” refers to molecular entities and compositions that do not produce an adverse, allergic or other untoward reaction when administered to a mammal, especially a human, as appropriate.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring, or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an antibody present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an antibody in a pharmaceutical composition of this invention may between about 1 mg/m² and 500 mg/m². However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

The invention will be further illustrated by the following figures and examples. However, these examples and figures should not be interpreted in any way as limiting the scope of the present invention.

### FIGURES

FIG. 1: Effect of intra-articular anti-GD3 monoclonal antibody injection in an experimental murine OA model. A) Experimental design. B) High-resolution imaging of bone structure (microCT). C-F) Analysis of the bone volume, ratio bone surface/bone volume (BS/BV), bone thickness and ratio bone surface/tissue volume (BS/TV) between isotype knee and α-GD3 knee (N=10) G-I) Confocal laser scanning microscopy, 3D reconstruction of cartilage. Analysis of the cartilage thickness, volume and ratio surface/volume (S/V) between isotype knee (N=10) and α-GD3 knee (N=10). P-values are calculated by the Mann-Whitney test (unpaired t test, two-tailed).

FIG. 2: Effect of intra-peritoneal injection of anti-GD3 monoclonal antibodies (R24) prevents bone remodeling in spontaneous age-related OA murine model. A-H

### EXAMPLE

### Material & Methods

### Human Cartilage Samples Banking

After signed consent, human OA cartilage banking was obtained from OA patients knee undergoing joint replacement surgery and declared to the national and local ethical committee: CEEA-LR-10042.

### Animal Experiments

12-week-old C57BL/6JCJ and 18-month-old male mice were obtained from Janvier Laboratory. Animal experiments were performed in accordance with the guidelines by the local ethics committee on animal research and care (saisine 1872-32846). After sacrifice, the knee joint was isolated, fixed in 4% paraformaldehyde at 4 °C for 2 days (IHC experiment) or for 7 days (bone and cartilage morphometric analysis).

### Experimental Osteoarthritis (OA) and Intra-Articular Injections of Antibodies

Experimental OA was induced in 12-weeks-old male mice according to the CIOA procedure. Under general anesthesia (isoflurane inhalation), the knee area was disinfected with ethanol and a small cut (a few millimeters) was performed in the cutaneous and subcutaneous tissue to visualize and to access the knee. Then, each mouse received an intra-articular injection of 5 μL (1IU) collagenase VII in the left knee to induce OA and 0.9% NaCl in the right knee as control, followed by a second injection two days later. Intra-articular injections of anti-GD3 (clone R24) or isotype (anti-IgG3) were performed in mice following the same procedure: 30 microgrammes in PBS of anti-GD3, or 30 microgrammes in PBS of isotype in the left knee at day 7, followed by a second injection 3 days later. For 18-month-old male mice, intra-peritoneal injections of anti-GD3 (clone R24) or isotype (anti-IgG3) were performed in mice: 150 microgrammes in PBS of anti-GD3, or 150 microgrammes in PBS of isotype, six times every 2 weeks.

### Safranin-O/Fast Green Staining

For cartilage analysis, mouse knees were fixed in 4% paraformaldehyde at 4 °C for 48 h, washed in PBS, and then processed for routine histology. Knees were decalcified in 14% EDTA/PBS for three weeks and then paraffin-embedded. Tissue sections (5 μm) were rehydrated through a gradient of ethanol and xylene. Sections were then stained with Safranin-O/Fast Green solution to evaluate cartilage degradation. Cartilage damage was analyzed using an arbitrary score of 0-30, based on the OARSI cartilage OA grading system histopathology, and modified by van den Berg for the assessment of murine knee joints (grading scale of 0-6 for the severity of cartilage destruction and of 0-5 for the extent of damaged cartilage surface).

### Immunohistochemistry (IHC) on OA Human and Mice Knee Joint Cartilage

Sections were embedded in paraffin and cut into sections (5 μm). The sections were deparaffinized in xylene and rehydrated in ethanol. Then, tissue sections were incubated with pepsin (DAKO, 40 mh/mL) for 30 min for antigen retrieval, permeabilized with 0.3% Triton X-100 PBS/BSA 3% during 3 min. 1% H202 for 10 min followed by incubation in PBS/BSA 3% for 30 min. Incubation with primary antibodies against GD3 (anti-GD3, 1:200, #MAB2053 Merck), p16INK4a (anti-p16INK4a, 1:200, #250804 ABBIOTECH) and p15 (anti-p15, 1:200, #orb213719 BIORBYT) were performed over-night at +4 °C. Sections were rinsed with PBS, then treated with Polink-2 Plus HRP Broad with DAB detection kit. Images were taken with Nanozoomer Digital Pathology (Hamamatsu) and number of positives cells were analyzed and quantified with the software QuPath.

### Bone Parameter Analyses

Hind leg knee were dissected to remove smooth tissues and scanned in a micro-CT scanner SkyScan 1176 (Bruker, Belgium, 0.5 mm aluminum filter, 45 kV, 500 μA, 18 μm resolution, 0.5° rotation angle). Scans were reconstructed using CTAn v1.9, Nrecon v1.6 (Bruker, Belgium) and a three-dimensional (3D) model visualization software program (CTVol v2.0). Misalignment compensation, ring artifacts and beam-hardening were adjusted to obtain the correct re-construction of each paw. Bone degradation was quantified in subchondral bone and the epiphysis region of the medial and/or lateral plateau for each tibia (CTAn software, Bruker, Belgium). Reconstructed 3D images of joints were obtained using the Avizo software (Avizo Lite 9.3.0, FEI, France).

### Cartilage Structure Quantification by Confocal Laser Scanning Microscopy

Articular cartilage of tibia medial plateau was scanned through their depth in XYZ-mode, with a confocal laser scanning microscope (CLSM; TCS SP5-II, Leica Microsystems, Nanterre, France) with a voxel size of 6 μm, a 5× dry objective and a UVlaser light source (1¼ 405 nm). Stacks of images were then done and analyzed to quantitatively evaluate several parameters of articular cartilage. Assessment of cartilage morphometric parameters was performed in medial plateau of each tibia using Avizo software (FEI Visualization Sciences Group, Lyon).

### Gene Expression Analysis by Real-Time Quantitative PCR

For qPCR experiments, total RNA was extracted from grinding frozen joint samples using Phenol/Chloroform method. RNA quality was checked by spectral analysis (A260/280 nm), and then samples were stored at −80 °C. Reverse transcription was performed using the M-MLV reverse transcriptase (Invitrogen; 28025013; 5 U/μL final concentration), 500 ng total RNA, a random hexamer primer (Thermo Scientific, GER; S0142; 10 ng/L final concentration), and dNTPs (Roche, CH; 1 277 049; 5 mM final concentration) in M-MLV reverse transcriptase buffer (Invitrogen; 18057-018), for a total volume of 20 μL. SYBR Green-based quantitative PCR was performed using the LightCycle® 480 SYBR Green I master reaction mix (Roche, 04707516001), 10 ng of cDNA, and the LightCycler 480 real-time PCR system (Roche) (40 cycles of amplification). Raw data (Ct values) were analyzed using the comparative Ct method. Gene expression data were calculated as relative to the expression of housekeeping gene, Rps9 (2\hat{ }-deltaCT method).

### Statistical Analysis

All data are presented as the median or mean±SEM. The Student's t-test was used for comparisons between experimental groups. For Spearman correlation, coefficient (r) was estimated to determine the linear association between 2 markers. Results were interpreted according to the degree of association as strong (r=0.5-1), moderate (r=0.5-0.7), or low (r=0.3-0.5) after taking significant correlation values into consideration; p-values <0.05 were considered significant (*p<0.05). Data were analyzed using the Prism software v9 (GraphPad Software Inc.).

### Results

GD3 Expression Correlates with p16INK4a Positive Senescent Cells and Increases with Cartilage Degeneration in OA Patients

We first wanted to determine the expression of GD3, a proposed senescence hallmark, in OA cartilage patients. We relied on declared tissue banking of tibial plateau human OA cartilage samples classified in two groups (low and high score) according to their cartilage degradation levels using the modified Mankin score and histological Safranin-O/Fast green staining. We evaluated by immunohistochemistry the presence of GD3 and p16INK4a positive cells in these OA cartilage samples (Data not shown). The quantification revealed a higher percentage of GD3 positive cells and p16INK4a positive cells with advanced Mankin score (Data not shown). In addition, we showed a significant correlation between GD3 positive chondrocytes and p16INK4a positive chondrocytes, supporting that GD3 positive cells are effectively senescent (Data not shown).

GD3 Expression Correlates with p21^{CIP1} and p15^{INK4b} Positive Senescent Cells and Increases with Cartilage Degeneration in Induced OA Murine Model

To reveal GD3 expression in experimental induced OA murine model, we relied as described on the intra-articular injection of collagenase VII (CiOA) into the joint of wild-type 3 months-old mice (Data not shown). At day 14, 28 and 42 post-injections, mice were sacrificed. We classified levels of cartilage degeneration in this model using the Safranin-O/Fast green staining and OA score as previously published. In the collagenase-injected joint compared to the NaCl control knee, we determined by immunostaining the presence of GD3 and two senescence-associated cell cycle inhibitors (p21^{CIP1} and p15^{INK4b}) positive cells within the cartilage along with progressive cartilage degeneration (Data not shown). Histological OA scoring and quantification of the percentage positive cells shows a significant correlation between GD3 positive cells and cartilage degeneration (Data not shown). Furthermore, we could reveal a significant correlation between GD3 positive chondrocytes and p21^{CIP1} or p15^{INK4b} positive cells (Data not shown).

### Intra-Articular Injection of Anti-GD3 Monoclonal Antibodies (R24) Prevents Bone Remodeling in Induced OA Murine Model

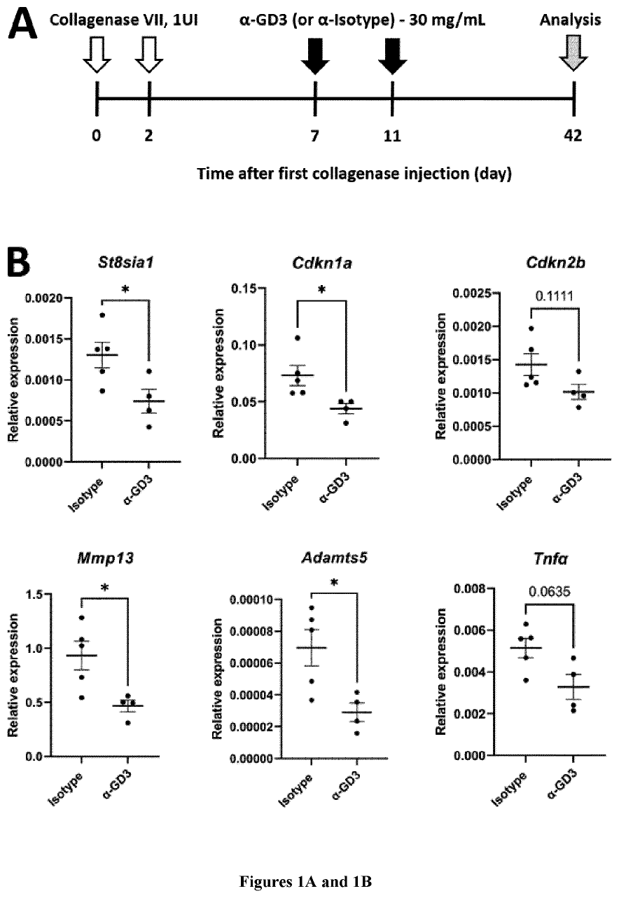

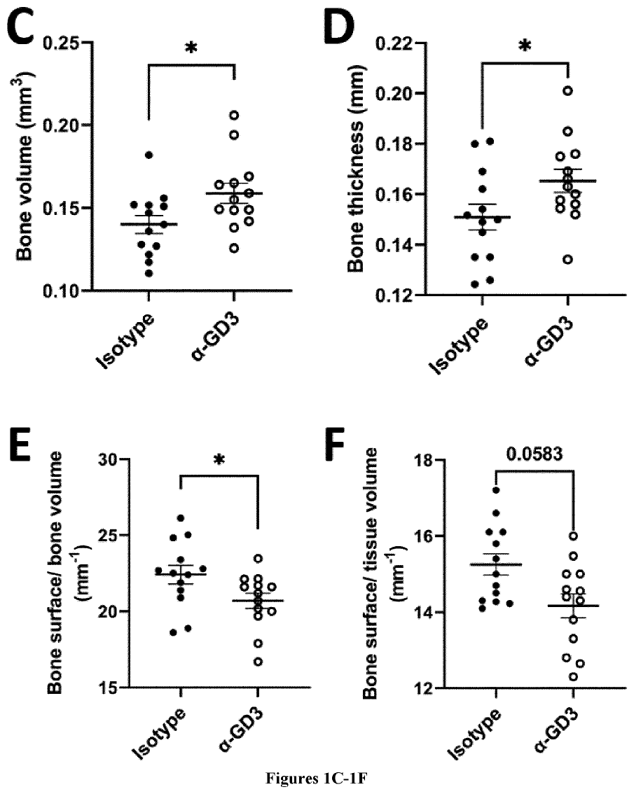

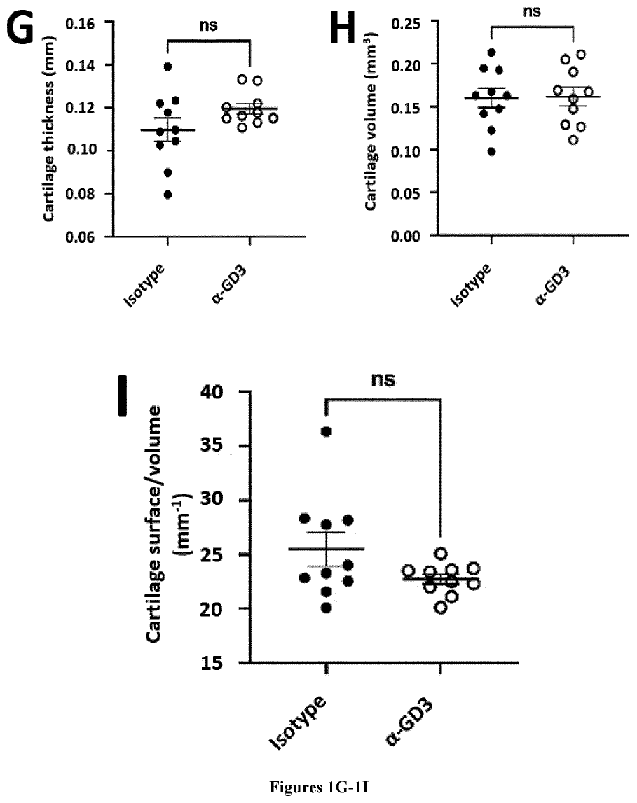

We next wanted to investigate whether an immunotherapy targeting GD3 marker can be efficient as OA therapy. For that we relied on a blocking monoclonal antibody against GD3 (anti-GD3 or isotype IgG3 control) injected into the joint twice on 3-month-old CIOA mice, (day 7 and 11) in the right OA knee (FIG. 1A). At day 42 after sacrifice, gene expression analysis from whole treated joint knee shows that St8sia1, and markers such as Cdkn1a, Cdkn2b, Mmp13, Adamts5 and Tnfα decrease with therapy compare to the Isotype group (FIG. 1B). This result confirms that anti-GD3 monoclonal antibody is able to target cells expressing senescence associated markers. In addition, murine joints were analyzed by microcomputed tomography (CT) and confocal laser scanning microscope (CLSM), for bone and cartilage structures respectively. The in vivo anti-GD3 articular injection induces a significant higher bone volume (FIG. 1C), higher bone thickness (FIG. 1D), lower ratio bone surface/bone volume (FIG. 1E) and almost significant lower ratio bone surface/tissue volume compared to control group (FIG. 1F). This reveals an anti-GD3 antibody protective effects against bone remodeling that characterized OA development and progression. However, analysis of cartilage structure by CLSM revealed no significant impact of anti-GD3 treatment compared to isotype control group (FIGS. 1G-1I).

### Intra-Peritoneal Injection of Anti-GD3 Monoclonal Antibodies (R24) Prevents Bone Remodeling in Spontaneous Age-Related OA Murine Model

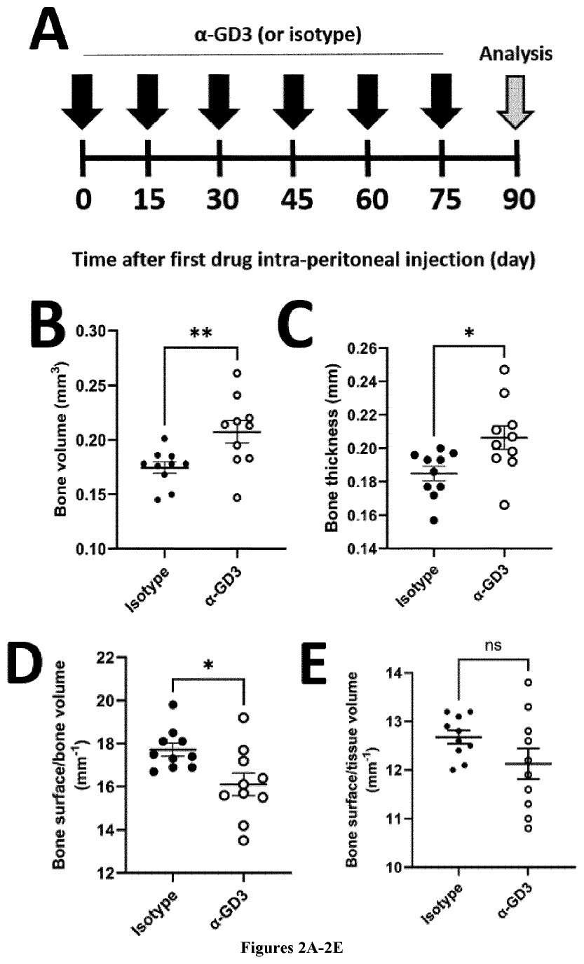

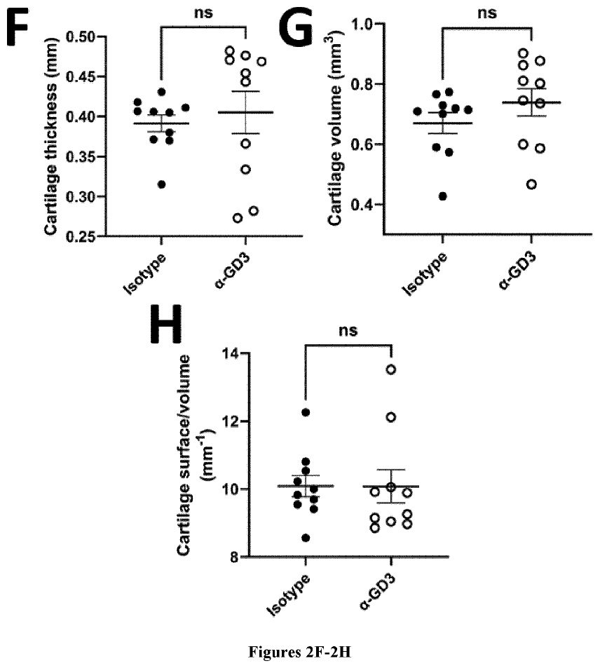

Finally, we wanted to investigate whether an immunotherapy targeting GD3 marker can be efficient as OA therapy in old mice. For that we relied on the same blocking monoclonal antibody against GD3 (anti-GD3 or isotype IgG3 control) injected into the peritoneum six times every 2 weeks on 18-month-old male mice (FIG. 2A). At day 90 after the first injection, and after sacrifice, murine joints were analyzed by microcomputed tomography (CT) and confocal laser scanning microscope (CLSM), for bone and cartilage structures respectively. The in vivo anti-GD3 peritoneal injection induces a significant higher bone volume (FIG. 2B), higher bone thickness (FIG. 2C), lower ratio bone surface/bone volume (FIG. 2D) but no changes in the ratio bone surface/tissue volume compared to control group (figure E). This reveals an anti-GD3 antibody protective effects against bone remodeling that characterized spontaneous OA deterioration. As in young CiOA male mice, analysis of cartilage structure by CLSM revealed no significant impact of anti-GD3 treatment compared to isotype control group (FIGS. 2F-2H).

### REFERENCES

Throughout this application, various references describe the state of the art to which this invention pertains. The disclosures of these references are hereby incorporated by reference into the present disclosure.

1. A method for treating osteoarthritis in a subject in need thereof, comprising administering to said subject a therapeutically effective amount of a GD3 inhibitor.

2. The method according to claim 1, wherein the GD3 inhibitor is an antibody.

3. The method according to claim 2, wherein the GD3 inhibitor is a monoclonal antibody.

4. The method according to claim 1, wherein the GD3 inhibitor is a small organic molecule.

5. The method according to claim 1, wherein the GD3 inhibitor is a polypeptide.

6. The method according to claim 1, wherein the GD3 inhibitor is an aptamer.

7. The method according to claim 1, wherein the GD3 inhibitor is an oligonucleotide.

8. The method according to claim 1, wherein the GD3 inhibitor is a CAR-T cell.

9. The method according to claim 1, wherein the GD3 inhibitor is a CAR NK cell.

10. The method according to claim 1, wherein the inhibitor of GD3 is administered to the subject in combination with a standard treatment.

11. The method according to claim 1, wherein the osteoarthritis is metabolic osteoarthritis.

12. The method according to claim 1, wherein the osteoarthritis is post-traumatic osteoarthritis.
